(19) United States
(12) Patent Application Publication
(10) Pub. No.: US 2018/0237691 A1
(43) Pub. Date: Aug. 23, 2018

(54) PRINTING INK AND ELECTRONIC DEVICE MANUFACTURED BY PRINTING WITH SAME

(71) Applicant: Guangzhou ChinaRay Optoelectronic Materials Ltd., Guangzhou (CN)

(72) Inventors: Junyou PAN, Guangzhou (CN); Xi YANG, Guangzhou (CN)

(73) Assignee: Guangzhou ChinaRay Optoelectronic Materials Ltd., Guangzhou (CN)

(21) Appl. No.: 15/751,370

(22) PCT Filed: Jul. 5, 2016
(86) PCT No.: PCT/CN2016/088639

§ 371 Date: Feb. 8, 2018

(30) Foreign Application Priority Data
Aug. 14, 2015 (CN) 201510502380.3

(51) Int. Cl. C09K 11/88; C01B 19/00; C01B 17/20; G01N 33/533; C30B 7/14

(52) U.S. Cl. CPC C09K 11/883; C01B 19/007; C30B 7/14; G01N 33/533; C01B 17/20

(57) ABSTRACT

A printing ink comprising inorganic nano-materials. The provided printing ink comprises at least one inorganic nano-material, in particular, quantum dots, and at least one aryl ketone-based or aryl ether-based organic solvent. Also provided is an electronic device manufactured by printing with the printing ink, in particular, an electroluminescent device.

### CROSS-REFERENCES TO RELATED APPLICATIONS

This application is a national stage application of PCT Patent Application No. PCT/CN2016088639, filed on Jul. 5, 2016, which claims priority to Chinese Patent Application No. 201510502380.3, filed on Aug. 14, 2015, the content of all of which is incorporated herein by reference.

### FIELD OF THE INVENTION

The present invention relates to a printing ink comprising an inorganic nano-material, the printing ink comprises at least one inorganic nano-material, specifically, a quantum dot, and at least onearylketone-based or arylether-based organic solvent; the present invention further relates to an electronic device manufactured by printing with the printing ink, specifically, an electroluminescent device.

### BACKGROUND

A quantum dot is a nano-sized semiconductor material with a quantum confinement effect. When stimulated by a light or electricity, the quantum dot will emit a fluorescence with a specific energy. A color (an energy) of the fluorescence is determined by a chemical composition, a size and a shape of the quantum dot. Therefore, a control of the size and shape of the quantum dot may effectively control a plurality of electrical and optical properties thereof. Currently, every country is studying an application of the quantum dots in a full-color area, mainly in a display area.

Recently, an electroluminescent device (Quantum dot Light Emitting Diodes, QLED) with the plurality of quantum dots working as a light-emitting layer has been rapidly developed, and a device lifetime thereof has been greatly improved, as published by Peng et al. in Nature Vol515 96 (2015) and Qian et al., Nature Photonics Vol 9259 (2015). Under an electric field applied, the electroluminescent device injects an electron and an electron hole into a light-emitting layer respectively to recombine before emitting a light. A spin-coating technology is currently a main method used to form a quantum dot light-emitting layer film. However, the spin-coating technology is hard to apply to manufacturing a large-area electroluminescent device. In a contrast, an ink-jet printing may manufacture the quantum dot film in a large-area and a low-cost; compared to a traditional semiconductor manufacturing process, the ink-jet printing has a plurality of advantages including a low power consumption, a low water consumption, and environmental friendly, which is a production technology with a great advantage and potential. A viscosity and a surface tension are important parameters affecting a printing ink and a printing process thereof. A promising printing ink needs a proper viscosity and surface tension. At present, a plurality of companies has reported a plurality of quantum dot inks for printing:

Nanoco Technologies Ltd of British, has disclosed a method of manufacturing a printable ink comprising a plurality of nanoparticles (CN101878535B). A printable nanoparticle ink and a film containing the nanoparticles accordingly, were obtained by selecting a suitable ink substrate, such as a toluene and a dodecane selenol.

Samsung Electronics has disclosed a quantum dots ink for ink-jet printing (U.S. Pat. No. 8,765,014B2). The ink contains a quantum dots-material in a certain concentration, an organic solvent, and a plurality of alcohol polymer additives with a high viscosity. By printing the ink, a quantum dots film is obtained, and a quantum dot electroluminescent device is prepared.

QD Vision, Inc. has disclosed a quantum dot ink formulation, the formulation comprises a host material, a quantum dot material and an additive (US2010264371A1).

A plurality of other patents related to the quantum-dots inks for printing are: US2008277626A1, US2015079720A1, US2015075397A1, TW201340370A, US2007225402A1, US2008169753A1, US2010265307A1, US2015101665A1, and WO2008105792A2. In these disclosed patents, to control a plurality of physical parameters of the inks, all these quantum-dots inks are containing other additives, such as an alcoholic polymer. However, introducing the additives of polymer with an insulating property may reduce an charge transportation capacity of the film, and have a negative impact on an optoelectronic property of the device, thus may limit a wide application thereof in an area of optoelectronic device. Therefore, finding an organic solvent system with an appropriate surface tension and viscosity for dispersing the quantum dots is particularly important.

### BRIEF SUMMARY OF THE DISCLOSURE

According to the above described defects, the purpose of the present invention is providing a new printing ink comprising an inorganic nano-material, the printing ink comprises at least one inorganic nano-material, specifically, a quantum dot material, and at least one arylketone-based or arylether-based organic solvent; the present invention further provides an electronic device manufactured by printing with the printing ink, specifically, an optoelectronic device, and more specifically, an electroluminescent device.

In order to achieve the above mentioned goals, the technical solution of the present invention to solve the technical problems is as follows:

A printing ink, comprising an inorganic nano-material, specifically a quantum dot, and at least one aryl ketone-based or aryl ether-based organic solvent, wherein, the aryl ketone-based or aryl ether-based organic solvent has a boiling point over 200° C., and a viscosity in a range of 1 cPs to 100 cPs at 25° C., the aryl ketone-based or aryl ether-based organic solvent is able to be evaporated from a solvent system and forming an inorganic nano-material film.

Wherein the aryl ketone-based or aryl ether-based organic solvent has a surface tension in a range of 19 dyne/cm to 50 dyne/cm at 25° C.

Wherein the aryl ketone-based or aryl ether-based organic solvent has a structure shown as a general formula (I) and (II), respectively:

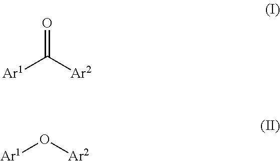

Wherein, an Ar1 and an Ar2 may be identical or different, and each is a substituted or unsubstituted aryl or heteroaryl ring system having 5 to 40 ring atoms;

the Ar1 and the Ar2 may be different, and one of them is a substituted or unsubstituted aryl or heteroaryl ring system having 5 to 40 ring atoms, another is a linear alkyl, alkoxy or thioalkoxy group having 1 to 20 carbon atoms, or a branched or cyclic alkyl, alkoxy or thioalkoxy group or a silyl group having 3 to 20 carbon atoms, or a substituted keto group having 1 to 20 carbon atoms, an alkoxycarbonyl group having 2 to 20 carbon atoms, an aryloxycarbonyl group having 7 to 20 carbon atoms, a cyano group (—CN), a carbamoyl group (—C(═O)NH2), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF3 group, a Cl, a Br, a F, a crosslinkable group, or a combination thereof.

Wherein the substituted or unsubstituted aryl or heteroaryl group, that both the Ar1 and Ar2 in the general formulas (I) and (II) are selected from, has a structure shown as a general formula below:

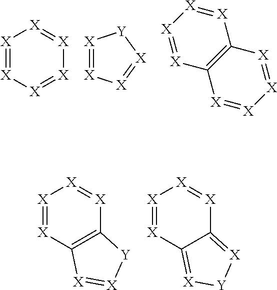

wherein,

X is CR1 or N;

Y is selected from CR2R3, SiR2R3, NR2 or, C(═O), S, or O;

R1, R2, R3 is an H, a D, or a linear alkyl, alkoxy or thioalkoxy group having 1 to 20 carbon atoms, or a branched or cyclic alkyl, alkoxy or thioalkoxy group or a silyl group having 3 to 20 carbon atoms, or a substituted keto group having 1 to 20 carbon atoms, an alkoxycarbonyl group having 2 to 20 carbon atoms, an aryloxycarbonyl group having 7 to 20 carbon atoms, a cyano group (—CN), a carbamoyl group (—C(═O)NH2), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF3 group, a Cl, a Br, anF, a crosslinkable group or a substituted or unsubstituted aryl or heteroaryl ring system having 5 to 40 ring atoms, or an aryloxy or a heteroaryloxy group having 5 to 40 ring atoms, or a combination thereof, wherein one or more groups of R1, R2, R3 may form a monocyclic or polycyclic aliphatic or aryl ring system by themselves or rings bonding with the groups.

Wherein the organic solvent is a single aryl ketone solvent or a mixture of a plurality of aryl ketone solvents or a mixture of aryl ketone solvents and other solvents; or the organic solvent is a single aryl ether solvent, or a mixture of a plurality of aryl ether solvents, or a mixture of aryl ether solvents and another solvents; or the organic solvent is a mixture of aryl ketone solvents and aryl ether solvents or the mixture further mixed with other solvents.

Wherein the organic solvents of the aryl ketones are 1-tetralone, 2-tetralone, acetophenone, propiophenone, benzophenone and a plurality of derivatives thereof.

Wherein the organic solvent of the aryl ether is 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl) benzene, 1,3-dipropylbenzene, 2,5-dimethoxytoluene, dibenzyl ether, 1, 2-dimethoxybenzene, glycidic phenyl ether and more.

Wherein the aryl ketone-based organic solvent is a tetralone or comprises at least 50% by weight of the tetralone and at least one other additional solvent.

Wherein the aryl ether-based organic solvent is a 3-phenoxytoluene or comprises at least 50% by weight of the 3-phenoxytoluene and at least one other additional solvent.

Wherein the inorganic nano-material is a quantum dot material, that is, a grain diameter thereof has a monodisperse size distribution, and a shape thereof may be selected from a plurality of different forms, including a sphere, a cube, a rod or a branched structure.

Wherein at least one luminescent quantum dot material is comprised, with a luminescence wavelength between 380 nm and 2500 nm.

Wherein the at least one inorganic nano-material is a binary or multiple semiconductor compound or a mixture thereof in Group IV, Group II-VI, Group II-V, Group III-V, Group III-VI, Group IV-VI, Group I-III-VI, Group II-IV-VI, Group II-IV-V of the Periodic Table.

Wherein the at least one inorganic nano-material is a perovskite nanoparticle material, specifically, a perovskite nanoparticle material, a metal nanoparticle material or a metal oxide nanoparticle material having a luminescent property, or a mixture thereof.

Wherein at least one organic functional material is further comprised, the organic functional material may be selected from a hole injection material (HIM), a hole transport material (HTM), an electron transport materials (ETM), an electron injection material (EIM), an electrons blocking material (EBM), a hole blocking material (HBM), a light emitter (Emitter), a host material (Host).

Wherein a weight ratio of the inorganic nano-material is 0.3%˜70%, a weight ratio of the organic solvent comprised based on aryl ketone or aryl ether is 30%˜99.7%.

An electronic device comprises a functional layer printed by the printing ink described above, wherein the aryl ketone-based or aryl ether-based organic solvent may be evaporated from the solvent system, and forming a film comprising the inorganic nano-materials.

Wherein the electronic device may be selected from a quantum dot light emitting diode (QLED), a quantum dot photovoltaic cell (QPV), a quantum dot light emitting electrochemical cell (QLEEC), a quantum dot field-effect transistor (QFET), a quantum dot light emitting field-effect transistor, a quantum dot Laser, a quantum dot sensor and more.

Benefits:

The present invention provides a printing ink comprising inorganic nanoparticles, the printing ink comprises at least one inorganic nano-material, specifically a quantum dot material, and at least one aryl ketone-based or aryl ether-based organic solvent. The printing ink provided by the present invention, may adjust a viscosity and a surface tension to a suitable range for facilitating printing, following a particular printing method, specifically, an ink jet printing, and forming a film having a uniform surface. While the aryl ketone-based or aryl ether-based organic solvent may be effectively removed by a post-treatment, including a heat treatment or a vacuum treatment, which facilitates assuring a performance of the electronic device. The present invention thus has provided a printing ink for preparing a high quality quantum dot film, which has provided a technical solution for printing a quantum dot electronic or optoelectronic device.

### BRIEF DESCRIPTION OF THE DRAWINGS

FIG. 1 illustrates a structural diagram of a preferred electroluminescent device according to the present prevention, wherein, 101 is a substrate, 102 is an anode, 103 is a hole injection layer (HIL) or a hole transport layer (HTL), 104 is a light emitting layer, 105 is an electron injection layer (EIL) or an election transport layer (ETL), 106 is a cathode.

### DETAILED DESCRIPTION

In order to make the purpose, technical solution and the advantages of the present invention clearer and more explicit, further detailed descriptions of the present invention are stated here, referencing to the attached drawings and some preferred embodiments of the present invention. It should be understood that the detailed embodiments of the invention described here are used to explain the present invention only, instead of limiting the present invention.

The present invention provides a printing ink, wherein comprising at least one inorganic nano-material and at least one aryl ketone-based or aryl ether-based organic solvent, wherein, the aryl ketone-based or aryl ether-based organic solvent has a boiling point over 200° C., and a viscosity in a range of 1 cPs to 100 cPs at 25° C., preferably, in a range of 1 cPs to 50 cPs, more preferably, in a range of 1 cPs to 30 cPs, and most preferably, in a range between 1.5 cPs and 20 cPs, the aryl ketone-based or aryl ether-based organic solvent may be evaporated from the solvent system and forming the inorganic nano-material film.

In a certain embodiment, the printing ink according to the present invention, wherein the arylketone-based or arylether-based organic solvent has a surface tension in a range of 19 dyne/cm to 50 dyne/cm at 25° C., preferably in a range of 20 dyne/cm to 40 dyne/cm, more preferably in a range of 22 dyne/cm to 35 dyne/cm, and most preferably, in a range of 25 dyne/cm to 33 dyne/cm.

In a preferred embodiment, the printing ink according to the present invention, wherein the aryl ketone-based organic solvent and the aryl ether-based organic solvent has a structure shown as a general formula of (I) and (II), respectively:

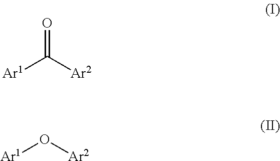

Wherein, Ar1 and Ar2 may be identical or different, and each is a substituted or unsubstituted aryl or heteroaryl ring system having 5 to 40 ring atoms;

The Ar1 and Ar2 may be different, and one of them is a substituted or unsubstituted aryl or heteroaryl ring system having 5 to 40 ring atoms, another is a linear alkyl, alkoxy or thioalkoxy group having 1 to 20 carbon atoms, or a branched or cyclic alkyl, alkoxy or thioalkoxy group or a silyl group having 3 to 20 carbon atoms, or a substituted keto group having 1 to 20 carbon atoms, an alkoxycarbonyl group having 2 to 20 carbon atoms, an aryloxycarbonyl group having 7 to 20 carbon atoms, a cyano group (—CN), a carbamoyl group (—C(═O)NH2), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF3 group, a Cl, a Br, a F, a crosslinkable group, or a combination thereof.

In a plurality of preferred embodiments, the Ar1 and Ar2 stated above may be identical or different, and each is a substituted or unsubstituted aryl or heteroaryl ring system having 5 to 20 ring atoms; the Ar1 and Ar2 may also be different, and one of them is a substituted or unsubstituted aryl or heteroaryl ring system having 5 to 20 ring atoms, another is a linear alkyl, alkoxy or thioalkoxy group having 1 to 10 carbon atoms, or a branched or cyclic alkyl, alkoxy or thioalkoxy group or a silyl group having 3 to 10 carbon atoms, or a substituted keto group having 1 to 10 carbon atoms, an alkoxycarbonyl group having 2 to 10 carbon atoms, an aryloxycarbonyl group having 7 to 10 carbon atoms, a cyano group (—CN), a carbamoyl group (—C(═O)NH2), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF3 group, a Cl, a Br, a F, a crosslinkable group, or a combination thereof.

In certain preferred embodiments, the organic solvent of the aryl ketones according to the general formula (I) or the aryl ethers according to the general formula (II), wherein Ar1 or Ar2 is selected from a substituted or unsubstituted aryl or heteroaryl group. An aryl group refers to a hydroxy containing at least one aryl ring, including a monocyclic group and a polycyclic ring system. A heteroaryl group refers to a hydroxy that contains at least one heteroaryl ring (heteroatoms contained), including a monocyclic group and a polycyclic ring system. The polycyclic rings may have two or more rings in which two carbon atoms are shared by two adjacent rings, that is, a condensed ring. In the polycyclic rings, at least one is aryl or heteroaryl.

In a plurality of more preferred embodiments, the substituted or unsubstituted aryl or heteroaryl group, that the Ar¹ and Ar² in the general formulas (I) and (II) are selected from, has a structure shown by a general formula below:

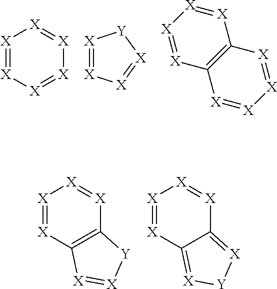

Wherein,

X is CR1 or NI;

Y is selected from CR2R3, SiR2R3, NR2 or, C(═O), S, or O;

R1, R2, R3 is an H, a D, or a linear alkyl, alkoxy or thioalkoxy group having 1 to 20 carbon atoms, or a branched or cyclic alkyl, alkoxy or thioalkoxy group or a silyl group having 3 to 20 carbon atoms, or a substituted keto group having 1 to 20 carbonatoms, an alkoxycarbonyl group having 2 to 20 carbon atoms, an aryloxycarbonyl group having 7 to 20 carbon atoms, a cyano group (—CN), a carbamoyl group (—C(═O)NH2), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF3 group, a Cl, a Br, anF, a crosslinkable group or a substituted or unsubstituted aryl or heteroaryl ring system having 5 to 40 ring atoms, or an aryloxy or a heteroaryloxy group having 5 to 40 ring atoms, or a combination thereof, wherein one or more groups of R1, R2, R3 may form a mono or polycyclic aliphatic or aryl ring system by themselves and/or rings bonding with the groups.

In a more preferred embodiment, R1, R2, R3 is an H, a D, or a linear alkyl, alkoxy or thioalkoxy group having 1 to 10 carbon atoms, or a branched or cyclic alkyl, alkoxy or thioalkoxy group or a silyl group having 3 to 10 carbon atoms, or a substituted keto group having 1 to 10 carbon atoms, an alkoxycarbonyl group having 2 to 10 carbon atoms, an aryloxycarbonyl group having 7 to 10 carbon atoms, a cyano group (—CN), a carbamoyl group (—C(═O)NH2), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF3 group, a Cl, a Br, anF, a crosslinkable group or a substituted or unsubstituted aryl or heteroaryl ring system having 5 to 20 ring atoms, or an aryloxy or a heteroaryloxy group having 5 to 20 ring atoms, or a combination thereof, wherein one or more groups of R1, R2, R3 may form a mono or polycyclic aliphatic or aryl ring system by themselves and/or rings bonding with the groups.

Specifically, suitableexamples of the aryl group include: benzene, naphthalene, anthracene, phenanthrene, perylene, tetracene, pyrene, benzopyrene, triphenylene, acenaphthene, fluorene and a plurality of derivatives thereof.

Specifically, suitable examples of the heteroaryl group include: furan, benzofuran, thiophene, benzothiophene, pyrrole, pyrazole, triazole, imidazole, oxazole, oxadiazole, thiazole, tetrazole, carbazole, pyrroloimidazole, pyrrolopyrrole, thienopyrrole, thienoisothiazole, furopyrrole, furofuran, thienofuran, benzisoxazole, benzisothiazole, benzimidazole, pyridine, pyrazine, pyridazine, pyrimidine, triazine, quinoline, isoquinoline, cinnoline, quinoxaline, phenanthridine, primary pyridine, quinazoline, quinazolinone, and a plurality of derivatives thereof.

More specifically, the suitable aryl group or heteroaryl group may be selected from (but not limited to) the groups listed below:

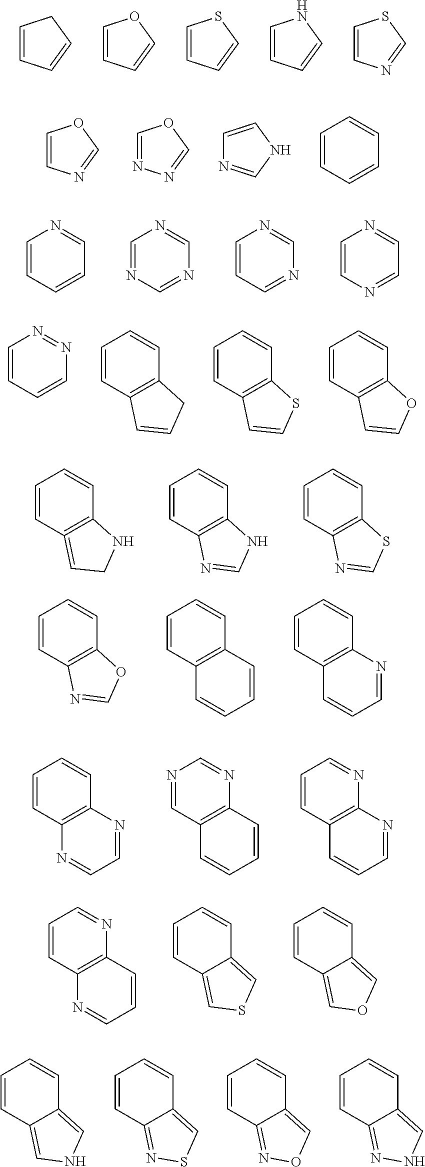

And, a substitution of R1 in the groups may be further conducted to obtain a plurality of substituted aryl or heteroaryl rings.

The solvent system based on the aryl ketones or aryl ethers according to the present invention, may effectively disperse the inorganic nanoparticles, especially the quantum dot materials, that is, acting as a new dispersion solvent in place of the dispersant conventionally used to dispersing inorganic nanoparticles such as a toluene, a Xylene, a chloroform, a chlorobenzene, a dichlorobenzene, an n-heptane and more.

Specifically, the organic solvent based on aryl ketones or aryl ethers, applied to dispersing inorganic nanoparticles, specifically quantum dots, when being selected, needs to take a boiling point parameter thereof into account. In certain preferred embodiments, the organic solvent based onaryl ketones or aryl ethers has a boiling point higher than 200° C., in certain embodiments, the organic solvent based on aryl ketones or aryl ethers has a boiling point higher than 250° C. In other preferred embodiments, the organic solvent based on aryl ketones or aryl ethers has a boiling point higher than 275° C. or higher than 300° C. Boiling points within these ranges are beneficial for preventing nozzle clogging of ink-jet print heads. The organic solvent based on aryl ketones or aryl ethers may be evaporated from the solvent system to form a film containing the inorganic nano-materials (or the quantum dots).

Specifically, the organic solvent based on aryl ketones or aryl ethers, applied to dispersing inorganic nanoparticles, specifically quantum dots, when being selected, needs to take the surface tension parameter thereof into account. A suitable surface tension parameter fits to a specific substrate and a specific printing method. For example, the ink jet printing, in a preferred embodiment, the organic solvent based on aryl ketones or aryl ethers has a surface tension in a range of about 19 dyne/cm to 50 dyne/cm at 25° C.; in a more preferred embodiment, the organic solvent based on aryl ketones or aryl ethers has a surface tension in a range of about 22 dyne/cm to 35 dyne/cm at 25° C.; in a most preferred embodiment, the organic solvent based on aryl ketones or aryl ethers, has a surface tension in a range of about 25 dyne/cm to 33 dyne/cm at 25° C.

Specifically, the organic solvent based on aryl ketones or aryl ethers, applied to dispersing inorganic nanoparticles, specifically quantum dots, when being selected, needs to take a viscosity parameter of the ink thereof into account. The viscosity may be adjusted through a plurality of different ways, including selecting a suitable organic solvent and a concentration of the nano-materials in the ink. Generally, a weight ratio of the inorganic nano-material contained in the printing ink according to the present invention is in a range of 0.3%˜70 wt %, preferably, in a range of 0.5%˜50 wt %, more preferably, in a range of 0.5%˜30 wt %, most preferably, in a range of 1%˜10 wt %. In a preferred embodiment, the ink of the organic solvent based on aryl ketones or aryl ethers has a viscosity lower than 100 cPs according to the above composition ratio; in a more preferred embodiment, the ink of the organic solvent based on aryl ketones or aryl ethers has a viscosity lower than 50 cPs according to the above composition ratio; in a most preferred embodiment, the ink of the organic solvent based on aryl ketones or aryl ethers has a viscosity in a range between 1.5 to 20 cPs according to the above composition ratio.

The inks obtained from the solvent system based on the aryl ketones or aryl esters satisfying the boiling points and surface tension parameters and viscosity parameters described above are capable of forming a film of the inorganic nanoparticles with a uniform thickness and a compositional property, specifically a film of the quantum dot.

In a certain embodiment, a solvent system that is well suited for the solvent system based on aryl ketones or aryl ethers, comprises a plurality of following systems: a single aryl ketone solvent, or a mixture of aryl ketone solvents, or a mixture of aryl ketone solvents and a plurality of other solvents; or a single aryl ether solvent or a mixture of a plurality of aryl ether solvents or a mixture of the aryl ether solvents and a plurality of other solvents; or a mixture of an aryl ketone solvent and an aryl ether solvent, or a mixture of the mixture further with other solvents.

In a certain embodiment, the aryl ketonesolvent is a tetralone solvent. Examples in the present invention related to the tetralone include 1-tetralone and 2-tetralone as below.

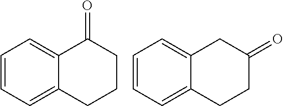

In certain embodiments, the tetralone solvent comprises a plurality of derivatives of the 1-tetralone and 2-tetralone, that is, the tetralone substituted by at least one substituent. These substituents include aliphatic, aryl, heteroaryl, halogen, and more. Specific examples are 2-(phenylepoxy) tetralone and 6-(methoxy) tetralone.

In a plurality of other embodiments, the aryl ketonesolventis acetophenone, propiophenone, benzophenone, and a plurality of derivatives thereof, such as 4-methylacetophenone, 3-methylacetophenone, 2-methylacetophenone, 4-methylpropiophenone, 3-methylpropiophenone and 2-methylpropiophenone.

In a plurality of other embodiments, the present invention may include some ketone solvents containing no aryl or heteroaryl groups, such as isophorone, 2,6,8-trimethyl-4-nonanone, camphor, fenchone.

In certain embodiments, the aryl ketone-based solvent system is a mixture, which may contain at least 50% of the aryl ketone solvent in the total weight of the solvent. Preferably, the solvent system includes the aryl ketone solvent at least 70% of the total weight of the solvent; more preferably, the solvent system includes the aryl ketone solvent at least 90% of the total weight of the solvent. And most preferably, the solvent system includes the aryl ketone solvent at least 99% of the solvent by weight, or consists essentially of, or entirely of the aryl ketone solvent.

In a preferred embodiment, the present invention relates to a printing ink, wherein the aryl ketone-based organic solvent is a 1-tetralone, or a mixture comprising at least 50% 1-tetralone by weight and at least one another additional solvent.

In another preferred embodiment, the present invention relates to a printing ink, wherein the aryl ketone-based organic solvent is a 2-tetralone, or a mixture comprising at least 50% 2-tetralone by weight and at least one another additional solvent.

In a certain embodiments, the aryl ether solvents suitable for the present invention are: 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl) benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, dimethoxybenzene, glycidic phenyl ether, dibenzyl ether, 4-tert-butyl anisole, trans-p-propenyl anisole, 1,2-dimethoxybenzene.

In a preferred embodiment, the aryl ether-based solvent is a 3-phenoxytoluene shown below:

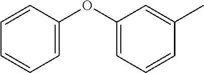

In certain embodiments, the aryl ether-based solvent system is a mixture that may contain at least 50% of the aryl ether solvent in the total weight of the solvent. Preferably, the aryl ether solvent included is at least 70% of the total weight of the solvent; more preferably, the aryl ether solvent included is at least 90% of the total weight of the solvent. Most preferably, the aryl ether-based solvent system comprises at least 99% by weight of the aryl ether solvent, or consists essentially or entirely of the aryl ether solvent.

In a preferred embodiment, the present invention relates to a printing ink, wherein the aryl ether-based organic solvent is a 3-phenoxytoluene or a mixture comprising at least 50% by weight of the 3-phenotoluene and at least one other solvent additionally.

In an embodiment, the 3-phenoxytoluene, or a mixture thereof with other ether solvents, or with other non-ether solvents, may be well suitable for the aryl ether-based solvent systems.

In a specific embodiment, the aryl ether-based solvent system may comprise 3-phenoxytoluene in an amount of at least 50% of a total weight of the solvent. Preferably, the solvent includes the 3-phenoxytoluene at least 70% of the total weight of the solvent; more preferably, the solvent includes the 3-phenoxytoluene at least 90% of the total weight of the solvent. Most preferably, the aryl ether-based solvent system comprises 99% by weight of the 3-phenoxytoluene, or consists essentially of 3-phenoxytoluene or consists entirely of 3-phenoytoluene.

In another embodiment, the printing ink further comprises another organic solvent. Examples on the organic solvent, include but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetralin, naphthalene alkane, indene, and/or a mixture thereof.

The printing ink may contain additionally one or more components such as a surface-active compound, a lubricant, a wetting agent, a dispersant, a hydrophobing agent, an adhesive and more, to adjust the viscosity or a film-forming property, to improve an adhesion, and more.

The printing ink may be deposited to obtain the quantum dot film by a plurality of techniques. A suitable printing or coating technique includes, but not limited to, an inkjet printing, a nozzle printing, a typography, a screen printing, a dip-coating, a spin-coating, a blade coating, a roller printing, a reverse-roll printing, a offset lithography printing, a flexography, a web printing, a spray coating, a brush coating or a pad printing, a slot-die coating and more. A preferred printing technique is gravure, a nozzle printing and an ink jet printing. For more information on the printing techniques and associated ink requirements thereof, such as a solvent and a concentration, a viscosity, etc., it may be referenced to Handbook of Print Media: Technologies and Production Methods, ISBN 3-540-67326-1, edited by Helmut Kipphan. In general, a different printing technology has a different character requirement for the ink used. For example, a printing ink suitable for the ink-jet printing needs to regulate a surface tension, a viscosity, and a wettability of the ink so that the ink may be squirted well through a nozzle at a printing temperature (such as a room temperature, 25° C.) instead of being dried out on the nozzle or blocking the nozzle, or form a continuous, smooth and defect-free film on a specific substrate.

The printing ink according to the present invention contains at least one inorganic nano-material.

In a certain plurality of embodiments, an average grain size of the inorganic nano-material is in a range about 1 to 1000 nm. In a certain preferred embodiment, the average grain size of the inorganic nano-material is in a range about 1 to 100 nm. In a certain more preferred embodiment, the average grain size of the inorganic nano-material is in a range about 1 to 20 nm, and most preferably, in a range of 1 to 10 nm.

The inorganic nano-material may be selected from a plurality of different shapes, including but not limited to a plurality of different nano-topographies including a sphere, a cube, a rod, a disk or a branched structure, and a mixture of various shaped particles.

In a preferred embodiment, the inorganic nano-material is a quantum dot material, having a very narrow and monodisperse size distribution, that is, a difference in dimension between the particles is very small. Preferably, the monodisperse quantum dots have a root mean square deviation (RMSD) in dimension less than 15% rms; more preferably, the monodisperse quantum dots have a RMSD in dimension less than 10% rms; and most preferably, the monodisperse quantum dots have a RMSD in dimension less than 5% rms.

In a plurality of preferred embodiments, the inorganic nano-material is an inorganic semiconductor material.

In another preferred embodiment, the inorganic nano-material is a luminescent material.

In a certain preferred embodiment, the luminescent inorganic nano-material is a quantum dot luminescent material.

In general, a luminescent quantum dot may emit a light at a wavelength between 380 nm and 2500 nm. For example, it has been found that, an emission wavelength of the quantum dot having a CdS core lies in a range of about 400 to 560 nm; an emission wavelength of a quantum dot having a CdSe core lies in a range of about 490 to 620 nm; an emission wavelength of a quantum dot having a CdTe core lies in a range of about 620 nm to 680 nm; an emission wavelength of a quantum dot having an InGaP core lies in a range of about 600 nm to 700 nm; an emission wavelength of a quantum dot having a PbS core lies in a range of about 800 nm to 2500 nm; an emission wavelength of a quantum dot having a CuInGaS core lies in a range of about 600 nm to 680 nm; an emission wavelength of a quantum dot having a ZnCuInGaS core lies in a range of about 500 nm to 620 nm; an emission wavelength of a quantum dot having a CuInGaSe core lies in a range of about 700 nm to 1000 nm;

In a preferred embodiment, the quantum dot material comprises at least one capable of emitting a blue light having an emission peak wavelength of 450 nm to 460 nm or a green light having an emission peak wavelength of 520 nm to 540 nm, or a red light having an emission peak wavelength of 615 nm to 630 nm, or a mixture thereof.

The quantum dots contained may be selected with a specific chemical composition, topography, and/or size, to achieve emitting the light in a desired wavelength under an electrical stimulation. A relationship between a luminescent property of a quantum dot and a chemical composition, topography and/or size thereof may be found in Annual Review of Material Science, 2000, 30, 545-610; Optical Materials Express, 2012, 2, 594-628; Nano Res., 2009, 2, 425-447. Entire contents of the patent documents listed above are hereby incorporated by reference.

A narrow grain size distribution of the quantum dots enables the quantum dots to have a narrower emission spectra (J. Am. Chem. Soc., 1993, 115, 8706; US20150108405). In addition, according to a different chemical composition and structure employed, a size of the quantum dots must be adjusted accordingly within the size range described above, to achieve the luminescence properties of a desired wavelength.

Preferably, the luminescent quantum dot is a semiconductor nanocrystal. In one embodiment, a size of the semiconductor nanocrystals is in a range of about 5 nm to about 15 nm. In addition, according to the different chemical composition and structure employed, the size of the quantum dots must be adjusted accordingly within the size range described above, to achieve the luminescence properties of a desired wavelength.

The semiconductor nanocrystal includes at least one semiconductor material, wherein the semiconductor material may be selected from a binary or multiple semiconductor compound or a mixture thereof, in group IV, group II-VI, group II-V, group III-V, group III-VI, group IV-VI, groups-III-VI, group II-IV-VI, group II-IV-V of a Periodic Table. Specifically, examples of the semiconductor material includes, but are not limited to, a group IV semiconductor compound, composed by a single elemental Si, Ge, C and a binary compound SiC, SiGe; a group II-VI semiconductor compound, consisting of a plurality of binary compounds including CdSe, CdTe, CdO, CdS, CdSe, ZnS, ZnSe, ZnTe, ZnO, HgO, HgS, HgSe, HgTe, a plurality of ternary compounds including CdSeS, CdSeTe, CdSTe, CdZnS, CdZnSe, CdZnTe, CgHgS, CdHgSe, ZnSeS, ZnSeTe, ZnSTe, HgSeS, HgSeTe, HgSTe, HgZnS, HgSeSe, and a plurality of quaternary compounds including CgHgSeS, CdHgSeTe, CgHgSTe, CdZnSeS, CdZnSeTe, HgZnSeTe, HgZnSTe, CdZnSTe, HgZnSeS; a group III-V semiconductor compound, consisting of a plurality of binary compounds including AlN, AlP, AlAs, AlSb, GaN, GaP, GaAs, GaSb, InN, InP, InAs, InSb, a plurality of ternary compounds including AlNP, AlNAs, AlNSb, AlPAs, AlPSb, GaNP, GaNAs, GaNSb, GaPAs, GaPSb, InNP, InNAs, InNSb, InPAs, InPSb, a plurality of quaternary compounds including GaAlNAs, GaAlNSb, GaAlPAs, GaInNP, GaInNAs, GaInNSb, GaInPAs, GaInPSb, InAlNP, InAlNAs, InAlNSb, InAlPAs, InAlPSb, a group IV-VI semiconductor compound, consisting of a plurality of binary compounds including SnS, SnSe, SnTe, PbSe, PbS, PbTe, a plurality of ternary compounds including SnSeS, SnSeTe, SnSTe, SnPbS, SnPbSe, SnPbTe, PbSTe, PbSeS, PbSeTe, and a plurality of quaternary compounds including SnPbSSe, SnPbSeTe, SnPbSTe.

In a preferred embodiment, the luminescent quantum dots comprise semiconductor compounds of groups II-VI, preferably selected from CdSe, CdS, CdTe, ZnO, ZnSe, ZnS, ZnTe, HgS, HgSe, HgTe, CdZnSe, and any combinations thereof. In a proper embodiment, since a synthesis of CdSe is relatively mature, the material is thus used as a luminescent quantum dot for a visible light.

In another preferred embodiment, the luminescent quantum dots comprise semiconductor compounds of groups III-V, preferably selected from InAs, InP, InN, GaN, InSb, InAsP, InGaAs, GaAs, GaP, GaSb, AlP, AlN, AlAs, AlSb, CdSeTe, ZnCdSe and any combinations thereof.

In another preferred embodiment, the luminescent quantum dots comprise semiconductor compounds of group IV-VI, preferably selected from PbSe, PbTe, PbS, PbSnTe, Tl2SnTe5, and any combinations thereof.

In a preferred embodiment, the quantum dot is a core-shell structure. Each of both the core and the shell comprises one or more semiconductor materials, either identical or different, respectively.

The core of the quantum dots may be selected from a binary or multiple semiconductors compound in the group IV, group II-VI, group II-V, group III-V, group III-VI, group IV-VI, group I-III-VI, group II-IV-VI, group II-IV-V of the Periodic Table. Specifically, embodiments on the core of the quantum dots include but not limited to ZnO, ZnS, ZnSe, ZnTe, CdO, CdS, CdSe, CdTe, MgS, MgSe, GaAs, GaN, GaP, GaSe, GaSb, HgO, HgS, HgSe, HgTe, InAs, InN, InSb, AlAs, AlN, AlP, AlSb, PbO, PbS, PbSe, PbTe, Ge, Si, or an alloy or a mixture of any combinations thereof.

The shell of the quantum dots may be selected from a plurality of semiconductor materials, identical or different from the core, preferably, from a plurality of semiconductor materials different from the core. The semiconductor materials may be used for the shell include a binary or multiple semiconductor compound in the group IV, group II-VI, group II-V, group III-V, group III-VI, group IV-VI, group I-III-VI, group II-IV-VI, group II-IV-V of a Periodic Table. Specifically, embodiments on the shell of the quantum dots include but not limited to ZnO, ZnS, ZnSe, ZnTe, CdO, CdS, CdSe, CdTe, MgS, MgSe, GaAs, GaN, GaP, GaSe, GaSb, HgO, HgS, HgSe, HgTe, InAs, InN, InSb, AlAs, AlN, AlP, AlSb, PbO, PbS, PbSe, PbTe, Ge, Si, or an alloy or a mixture of any combinations thereof.

The quantum dot with the core-shell structure, wherein, the shell may include a single-layer or multi-layer structure. The shell includes one or more semiconductor materials that are identical or different from the core. In a preferred embodiment, the shell has a thickness of about 1 to 20 layers. In a more preferred embodiment, the shell has a thickness of about 5 to 10 layers. In certain embodiments, two or more shells are grown on a surface of the core of the quantum dot.

In a preferred embodiment, the semiconductor material used for the shell has a larger bandgap than the core. Preferably, the core-shell has an 1-type semiconductor heterojunction structure.

In another preferred embodiment, the semiconductor material used for the shell has a smaller bandgap than the core.

In a preferred embodiment, the semiconductor material used for the shell has an atomic crystal structure that is the same as or close to the core. Such a choice helps to reduce a stress between the core and the shell, while making the quantum dots more stable.

In a preferred embodiment, the quantum dots with the core-shell structure adopted are, but not limited to:

Red light: CdSe/CdS, CdSe/CdS/ZnS, CdSe/CdZnS and more,

Green light: CdZnSe/CdZnS, CdSe/ZnS and more,

Blue light: CdS/CdZnS, CdZnS/ZnS and more.

A preferred method for preparing the quantum dots is a colloidal growth method. In a preferred embodiment, a method for preparing a monodisperse quantum dot is selected from a hot-inject method and/or a heating-up method. The method for preparation is disclosed in a reference in Nano Res, 2009, 2, 425-447; Chem. Mater., 2015, 27 (7), pp 2246-2285. The entire contents of the documents listed above are hereby incorporated by reference.

In a preferred embodiment, the surface of the quantum dot contains a plurality of organic ligands. An organic ligand may control a growth of the quantum dots, control an appearance of the quantum dots and reduce a surface defect of the quantum dots, so as to improve a luminous efficiency and stability of the quantum dots. The organic ligand may be selected from a pyridine, a pyrimidine, a furan, an amine, an alkylphosphine, an alkylphosphine oxide, an alkylphosphonic acid or an alkylphosphinic acid, an alkylthiol and more. Specific examples of organic ligands include, but are not limited to, tri-n-octylphosphine, tri-n-octylphosphine oxide, trihydroxypropylphosphine, tributylphosphine, 3(dodecyl) phosphine, dibutyl phosphite, tributyl phosphite, octadecyl phosphite, trilauryl phosphite, didodecyl phosphite, triisodecyl phosphite, bis (2-ethylhexyl) phosphate, Tridecyl phosphate, hexadecylamine, oleylamine, octadecylamine, dioctadecylamine, octacosamine, bis (2-ethylhexyl) amine, octylamine, dioctylamine, Amine, dodecylamine, didodecylamine, didodecylamine, hexadecylamine, phenyl phosphate, hexyl phosphate, tetradecyl phosphate, octyl phosphate, n-octadecyl phosphate, propylene diphosphate, dioctyl ether, Diphenyl ether, octyl mercaptan, dodecyl mercaptan.

In another preferred embodiment, the surface of the quantum dot contains a plurality of inorganic ligands. The quantum dots protected by the inorganic ligands may be obtained by ligand exchange of organic ligands on the surface of quantum dots. Specifically, an embodiment on inorganic ligands includes but not limited to: S2-, HS—, Se2-, HSe—, Te2-, HTe—, TeS32-, OH—, NH2-, PO43-, MoO42-, and more. Examples on such an inorganic ligand quantum dot may refer to a document of J. Am. Chem. Soc. 2011, 133, 10612-10620; ACS Nano, 2014, 9, 9388-9402. All contents of the documents listed above are hereby incorporated for reference.

In certain embodiments, the surface of the quantum dots has one or more identical or different ligands.

In a preferred embodiment, a luminescence spectrum exhibited by a monodisperse quantum dot has a symmetrical peak shape and a narrow peak width at half height. Generally, the better a monodispersity of the quantum dots is, the more symmetrical a luminescence peak is, and the narrower the peak width at half height is. Preferably, the peak width at half height of the quantum dot is less than 70 nm; more preferably, the peak width at half height of the quantum dot is less than 40 nm; and most preferably, the peak width at half height of the quantum dot is less than 30 nm.

The quantum dots have a luminous quantum efficiency of 10%-100%. Preferably, the quantum dots have a luminous quantum efficiency of more than 50%; more preferably the quantum dots have a luminous quantum efficiency of more than 80%; most preferably, the quantum dots have a luminous quantum efficiency of more than 90%.

A plurality of other materials, techniques, methods, applications, and other information concerning quantum dots that may be useful in the present invention are described in a plurality of patent documents following: WO2007/117698, WO2007/120877, WO2008/108798, WO2008/105792, WO2008/111947, WO2007/092606, WO2007/117672, WO2008/033388, WO2008/085210, WO2008/13366, WO2008/063652, WO2008/063653, WO2007/143197, WO2008/070028, WO2008/063653, U.S. Pat. No. 6,207,229, U.S. Pat. No. 6,251,303, U.S. Pat. No. 6,319,426, U.S. Pat. No. 6,426,513, U.S. Pat. No. 6,576,291, U.S. Pat. No. 6,607,829, U.S. Pat. No. 6,861,155, U.S. Pat. No. 6,921,496, U.S. Pat. No. 7,060,243, U.S. Pat. No. 7,125,605, U.S. Pat. No. 7,138,098, U.S. Pat. No. 7,150,910, U.S. Pat. No. 7,470,379, U.S. Pat. No. 7,566,476, WO2006134599A1. All contents of the documents listed above are hereby incorporated for reference.

In another preferred embodiment, a luminescent semiconductor nanocrystal is a nanorod. A characteristics of the nanorods is different from a spherical nanocrystal. For example, the luminescence of a nanorod is polarized along a long rod axis while the luminescence of a spherical crystal is unpolarized (refer to Woggon et al., Nano Lett., 2003, 3, p 509). The nanorod has an excellent characteristic on an optical gain that makes them potentially useful as a laser gain material (refer to Banin et al., Adv. Mater. 2002, 14, p 317). Additionally, the luminescence of the nanorod may be switched on and off reversibly under a control of an external electric field (refer to Banin et al., Nano Lett. 2005, 5, p 1581). A plurality of these characteristics of the nanorods may be preferentially incorporated into a device of the present invention, in some cases. An example of preparing a semiconductor nanorod includes: WO03097904A1, US2008188063A1, US2009053522A1, KR20050121443A. All contents of the documents listed above are hereby incorporated for reference.

In other preferred embodiments, the printing ink according to the present invention, wherein, the inorganic nano-material is a nanoparticle material of perovskite, specifically, a luminescent nanoparticle material of perovskite.

The nanoparticle material of perovskite has a general formula of AMX3, wherein A includes an organic amine or an alkali metal cation, M includes a metal cation, X includes an oxyanion or a halogen anion. A specific embodiment includes but not limited to: CsPbCl3, CsPb(Cl/Br)3, CsPbBr3, CsPb(I/Br)3, CsPbl3, CH3NH3PbCl3, CH3NH3Pb(Cl/Br)3, CH3NH3PbBr3, CH3NH3Pb(I/Br)3, CH3NH3PbI3, and more. A plurality of examples on the nanoparticle material of perovskite may be referred to: NanoLett., 2015, 15, 3692-3696, ACS Nano, 2015, 9, 4533-4542; AngewandteChemie, 2015, 127(19):5785-5788, NanoLett., 2015, 15(4), pp 2640-2644, Adv. OpticalMater., 2014, 2, 670-678, The Journal of Physical Chemistry Letters, 2015, 6(3):446-450; J. Mater. Chem. A, 2015, 3, 9187-9193; Inorg. Chem. 2015, 54, 740-745, RSCAdv., 2014, 4, 55908-55911, J. Am. Chem. Soc., 2014, 136(3), pp 850-853, Part. Part. Syst. Charact. 2015, doi:10.1002/ppsc.201400214, Nanoscale, 2013, 5(19):8752-8780. All contents of the documents listed above are hereby incorporated for reference.

In another preferred embodiment, the printing ink according to the present invention, wherein, the inorganic nano-material is a metal nanoparticle material.

The metal nanoparticle material includes but not limited to: nanoparticles of Cr, Mo, W, Ru, Rh, Ni, Ag, Cu, Zn, Pd, Au, Os, Re, Ir and Pt. A species, a morphology and a synthesis method of the metal nanoparticle material commonly seen may refer to Angew. Chem. Int. Ed. 2009, 48, 60-103; Angew. Chem. Int. Ed. 2012, 51, 7656-7673; Adv. Mater. 2003, 15, No. 5, 353-389, Adv. Mater. 2010, 22, 1781-1804; Small. 2008, 3, 310-325; Angew. Chem. Int. Ed. 2008, 47, 2-46, and more, as well as all references thereof. All contents of the documents listed above are hereby incorporated for reference.

In another preferred embodiment, the inorganic nano-material has a property of charge transport.

In a preferred embodiment, the inorganic nano-material has a capability of an electron transport. Preferably, such an inorganic nano-material is selected from an n-type semiconductor material. An example of an n-type inorganic semiconductor material includes, but not limited to, a metal chalcogenide, a metal pnictide, or an elemental semiconductor, such as a metal oxide, a metal sulfide, a metal selenide, a metal telluride, a metal nitride, a metal phosphide, or a metal arsenide. Preferably, the n-type inorganic semiconductor material may be selected from: ZnO, ZnS, ZnSe, TiO2, ZnTe, GaN, GaP, AlN, CdSe, CdS, CdTe, CdZnSe, and any combinations thereof.

In certain embodiments, the inorganic nano-material has a hole transport capability. Preferably, such an inorganic nano-material is selected from a p-type semiconductor material. An inorganic p-type semiconductor material may be selected from: NiOx, WOx, MoOx, RuOx, VOx, and any combinations thereof.

In a certain embodiment, the printing ink according to the present invention comprises at least two or more kinds of inorganic nano-materials.

In a certain embodiment, the printing ink according to the present invention further comprises at least one organic functional material. As described above, an object of the present invention is preparing an electronic device from a solution, due to a solubility in an organic solution and an inherent flexibility thereof, an organic material may be incorporated into a functional layer of an electronic device in a certain case, and bringing a plurality of other benefits, such as enhancing a flexibility of the device, improving a performance of film-making and so on. Asa principle, all organic functional materials applied for OLEDs, include but not limited to, hole injection material (HIM), hole transport material (HTM), electron transport material (ETM), electron injection material (EIM), electron blocking material (EBM), hole blocking material(HBM), light emitter (Emitter) and host material (Host) may all be applied in the printing ink of the present invention. Various organic functional materials are described in detail, for example, in WO2010135519A1 and US20090134784A1. All contents of the documents listed above are hereby incorporated for reference.

The present invention further relates to an electron device, containing one or a plurality of functional films, wherein at least one layer of the film is prepared by the printing ink according to the present invention, specifically, through a method of printing or coating.

The film containing nanoparticles according to the present invention is prepared through a method of printing or coating. In a preferred embodiment, the film containing nanoparticles is prepared through a method of ink jet printing. An ink jet printer applied to printing the ink containing the quantum dots according to the present invention may be a printer already commercially available, which contains a drop-on-demand print head. Such a printer may be bought from Fujifilm Dimatix (Lebanon, N.H.), Trident International (Brookfield, Conn.), Epson(Torrance, Calif.), Hitachi Data systems Corporation (Santa Clara, Calif.), Xaar PLC (Cambridge, United Kingdom), and Idanit Technologies, Limited (Rishon Le Zion, Isreal). For example, the present invention may be printed by Dimatix materials Printer DMP-3000 (Fujifilm).

A suitable electronic device includes but not limited to: a quantum dot light emitting diode (QLED), a quantum dot photovoltaic cell (QPV), a quantum dot light emitting electrochemical cell (QLEEC), a quantum dot field-effect transistor (QFET), a quantum dot light emitting Laser, a quantum dot light emitting field-effect transistor, a quantum dot sensor and more.

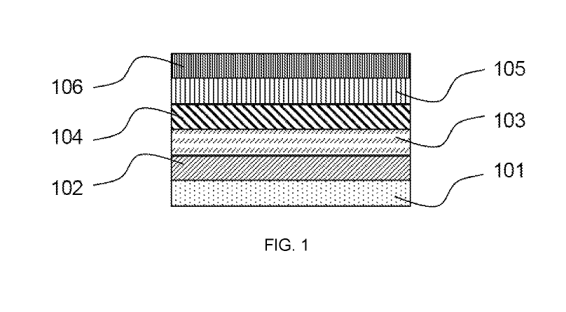

In a preferred embodiment, the electronic device listed above is an electroluminescent device, as shown in FIG. 1, the electroluminescent device comprises a substrate (101), an anode (102), at least a light emitting layer (104), a cathode (106). The substrate (101) may be opaque or transparent. A transparent substrate can be applied to making a transparent light-emitting component. Refer to, for example, Bulovic et al. Nature 1996, 380, p 29, and Gu et al., Appl. Phys. Lett. 1996, 68, p 2606. A substrate material may be rigid or elastic. The substrate may be a plastic, a metal, a semiconductor wafer or a glass. Preferably, the substrate has a smooth surface. A substrate without any surface defects is a particularly desirable choice. In a preferred embodiment, the substrate is selected from a polymer film or a plastic, which has a glass transition temperature Tg of 150° C. or higher, preferably over 200° C., more preferably over 250° C. and most preferably over 300° C. An example of the suitable substrate is a poly (ethylene terephthalate) (PET) or a polyethylene glycol (2,6-naphthalene) (PEN).

The anode (102) may comprise a conductive metal or a metal oxide, or a conductive polymer. The anode may easily inject an electron hole into the HIL or HTL or the light-emitting layer. In an embodiment, an absolute value of the difference between the a work function of the anode and an HOMO level or a valence band level of the p-type semiconductor material working as HIL or HTL is less than 0.5 eV, preferably less than 0.3 eV, and most preferably, less than 0.2 eV. An example of an anode material includes, but not limited to, Al, Cu, Au, Ag, Mg, Fe, Co, Ni, Mn, Pd, Pt, ITO, aluminum-doped zinc oxide (AZO) and more. Other suitable anode materials are known and may be readily selected by an ordinary skilled man in the art. The anode material may be deposited using any suitable techniques, such as a suitable physical vapor deposition method, including a radio frequency magnetron sputtering deposition, a vacuum thermal evaporation deposition, an e-beam deposition, and more.

In certain embodiments, the anode is a pattern structured. A patterned ITO conductive substrate is commercially available and may be applied to making devices according to the present invention.

The cathode (106) may comprise a conductive metal or a metal oxide. The cathode may easily inject an electron into the EIL or ETL or directly to the light-emitting layer. In an embodiment, an absolute value of the difference between the work function of the cathode and a LUMO level or a conduction band level of the n-type semiconductor material working as EIL or ETL or HBL is less than 0.5 eV, preferably less than 0.3 eV, and most preferably, less than 0.2 eV. Principally, all materials being able to be applied to a cathode of an OLED may be applied to the cathode materials according to the present invention. An example of a cathode material includes, but not limited to, Al, Au, Ag, Ca, Ba, Mg, LiF/Al, Mg or Ag alloy, BaF2/Al, Cu, Fe, Co, Ni, Mn, Pd, Pt, ITO and more. A cathode material may be deposited using any suitable techniques, such as a suitable physical vapor deposition method, including a radio frequency magnetron sputtering deposition, a vacuum thermal evaporation deposition, an e-beam deposition, and more.

The light emitting layer (104) contains at least one luminescent nano-material, with a thickness between 2 nm and 200 nm. In a preferred embodiment, a light-emitting device according to the present invention, wherein, the light-emitting layer thereof is prepared by printing the printing ink according to the present invention, while the printing ink contains the luminescent nano-material as described above, specifically a quantum dot.

In a preferred embodiment, the light-emitting device according to the present invention further comprises a hole injection layer (HIL) or a hole transport layer (HTL) (103), wherein comprising an organic HTM or an inorganic p-type material. In a preferred embodiment, HIL or HTL may be prepared by printing the printing ink of the present invention, wherein the printing ink contains an inorganic nano-material having a hole-transport ability, specifically a quantum dot.

In another preferred embodiment, the light emitting device according to the present invention further comprises an electron injection layer (EIL) or an electron transport layer (ETL) (105), wherein comprising the organic ETM as described above or the inorganic n-type material. In a preferred embodiment, an EIL or ETL may be prepared by printing the printing ink of the present invention, wherein the printing ink contains an inorganic nano-material having an electron-transport ability, specifically a quantum dot.

The present invention further relates to an application of the light-emitting device according to the present invention in various cases including but not limited to, a plurality of various display devices, a plurality of backlights, a plurality of illuminating light sources and more.

The present invention will be described below with reference to a plurality of preferred embodiments, however, the present invention is not limited to the following embodiments. It should be understood that a plurality of claims appended have summarized a scope of the present invention, and under a guidance of the concept of the present invention, a technical personnel in the art should recognize that, a certain changes to the disclosed embodiments are intended to be encompassed within the scope of the claims of the present disclosure.

### EMBODIMENTS

### Embodiment 1: Preparing a Blue Light Quantum Dot (CdZnS/ZnS)

Weigh 0.0512 g sulphur and an amount of 2.4 mLODE before putting into a 25 mL one-necked flask, then place the flask into an oil pan before heated to 80° C. to dissolve the sulphur, standby, hereinafter it is referred to solution 1; Weigh 0.1280 g Sulphur and an amount of 5 mLOA before putting into a 25 mLone-necked flask, and then place the flask into an oil pan before heated to 90° C. to dissolve the Sulphur, standby, hereinafter it is referred to solution 2; Weigh 0.1028 gCdO and 1.4680 g zinc acetate, and an amount of 5.6 mL OA, before putting into a 50 mL three-necked flask, then place the three-necked flask in a 150 mL heating mantle, while plugging both necks on sides with two rubber plugs, and a condenser is connected above, the flask is then connected to a double-tube, before heated to 150° C., and evacuated for 40 minutes, and then purged with nitrogen. Using a syringe to add 12 mL ODE into the three-necked flask, before heated to 310° C., followed by injecting rapidly 1.92 mL solution 1 by a syringe into the three-necked flask, count for 12 min, add 4 mL of the solution 2 by drops into the three-necked flask with a syringe right after the 12 min, with a dropping rate about 0.5 mL/min, wait for a reaction lasting 3 h, before stopping the reaction, and put the three-necked flask immediately in water to cool down to 150° C.;

Add an excess n-hexane into the three-necked flask, then transfer a liquid in the three-necked flask to a plurality of centrifuge tubes of 10 mL, centrifuge and remove a lower sediment, repeat for three times; add acetone to the solution after the treatment to produce a precipitate, centrifuge and remove a supernatant, with a precipitate left; followed by using an n-hexane to dissolve the precipitate, and adding acetone for precipitating, remove a supernatant after centrifuge, with a precipitate left, repeat for three times; finally, dissolve the precipitate with a toluene before transferring to a glass bottle for storage.

### Embodiment 2: Preparing a Green Light Quantum Dot (CdZnSeS/ZnS)

Weigh 0.0079 g selenium and 0.1122 g sulphur before putting into a 25 mL one-necked flask, and an amount of 2 mL TOP, purged with nitrogen, stir and standby, thereafter it is referred to solution 1; weight 0.0128 g CdO and 0.3670 g zinc acetate, and an amount of 2.5 mL OA before putting into a 25 mL three-necked flask, while plugging both necks on sides with two rubber plugs, and a condenser is connected above, the flask is then connected to a double-tube, followed by placing the three-necked flask into a 50 mL heating mantle, vacuumed and purged with nitrogen, before heated to 150° C., and vacuumed for 30 min, injected with 7.5 mL ODE, and heated to 300° C., followed by injecting rapidly 1 mL solution 1, count for 10 min; stop the reaction right after 10 min, and place the three-necked flask into water for cooling.

Add 5 mL n-hexane into the three-necked flask, then transfer the mixture in the three-necked flask into a plurality of centrifuge tubes of 10 mL, add acetone to produce a precipitate, and centrifuge. Take the precipitate, remove a supernatant, and dissolve the precipitate with n-hexane, add acetone to produce a precipitate, and centrifuge. Repeat for three times. And, at last, the participate is dissolved with a small amount of toluene, before transferring to a glass bottle for storage.

### Embodiment 3: Preparing a Red Light Quantum Dot (CdSe/CdS/ZnS)

Add 1 mmol CdO, 4 mmol OA and 20 mL ODE into a 100 mL three-necked flask, purged with nitrogen, heat to 300° C. and a precursor of Cd(OA)2 is formed. At such a temperature, inject rapidly 0.25 mL TOP with 0.25 mmol selenium powder dissolved. React for 90 seconds at such a temperature, a reaction solution grows for a 3.5 nm CdSe core. Add 0.75 mmol octylmercaptan into the reaction solution by drops at a temperature of 300° C., react for 30 min and a CdS shell with about 1 nm thickness is grown. Followed by adding 4 mmol Zn(OA)2 and 2 ml TBP with 4 mmol sulphur powder dissolved into the reaction solution by drops, to grow a ZnS shell (about 1 nm). The reaction lasts for 10 minutes before cooling to a room temperature.

Add 5 mL n-hexane into the three-necked flask, then transfer the mixture in the three-necked flask into a plurality of centrifuge tubes of 10 mL, add acetone to produce a precipitate, and centrifuge. Take the precipitate, remove a supernatant, and dissolve the precipitate with n-hexane, add acetone to produce a precipitate, and centrifuge. Repeat for three times. And, at last, the participate is dissolved with a small amount of toluene, before transferring to a glass bottle for storage.

### Embodiment 4: Preparing a ZnO Nanoparticle

Dissolve 1.475 g zinc acetate in 62.5 mL methanol, and obtain a solution 1. Dissolve 0.74 g KOH in 32.5 mL methanol, and obtain a solution 2. Heat the solution 1 up to 60° C. and stir vigorously. Add the solution 2 by drops into the solution 1 using a sampler. After completed, the mixture is kept stirring for 2 hours at 60° C. Remove the heat source and leave the solution system undisturbed for 2 hours. Under a centrifugation condition of 4500 rpm, 5 min, wash the reaction solution by centrifugation three times or more. And the white solid finally obtained is the ZnO nanoparticle with a diameter of about 3 nm.

### Embodiment 5: Preparing the Printing Ink with the Quantum Dots Containing the 1-Tetralone

A plurality of organic solvents according to the present invention, wherein a boiling point and a rheological parameter thereof are shown in Table 1 below.

| | | Boiling | Surface | |
|---|---|---|---|---|
| | | point | tension@RT | Viscosity@RT |
| Organic solvent | Structure | (° C.) | (dyne/cm) | (cPs) |
| 1-tetralone | 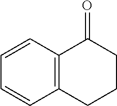 [SMILES: O=C1CCCc2ccccc21] | 256 | 42 | 8.6 |
| 3-phenoxytoluene | 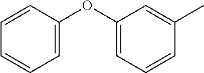 | 272 | 37.4 | 5 |
| Acetophenone | 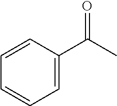 | 202 | 39 | 1.6 |
| 1-tetralone and | 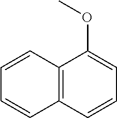 | 270 | 43 | 7.2 |

Put a stirrer into a vial, clean it up before transferring to a glove box. In the vial, prepare 9.5 g 1-tetralone. Separate out the quantum dots from the solution with acetone, centrifuge and obtain a solid of quantum dots. Weigh 0.5 g the solid of the quantum dots from the glove box, add to the solvent system in the vial, stir and mix. Keep stirring at a temperature of 60° C. until the quantum dots fully dispersed, cool to the room temperature. Membrane Filtrate the quantum dot solution obtained through a 0.2 μm PTFE filter. Seal and store.

### Embodiment 6: Preparing the Printing Ink with the Quantum Dots Containing the 3-Phenoxytoluene

Put a stirrer into a vial, clean it up before transferring to a glove box. In the vial, prepare 9.5 g of the 3-phenoxytoluene. Precipitate the quantum dots from the solution with acetone, centrifuge and obtain a solid of quantum dots. Weigh 0.5 g the solid of the quantum dots from the glove box, add to the solvent system in the vial, stir and mix. Keep stirring at a temperature of 60° C. until the quantum dots fully dispersed, cool to the room temperature. Membrane Filtrate the quantum dot solution obtained through a 0.2 μm PTFE filter. Seal and store.

### Embodiment 7: Preparing the Printing Ink with the Quantum Dots Containing a Mixture of the 1-Tetralone and 3-Phenoxytoluene

Put a stirrer into a vial, clean it up before transferring to a glove box. In the vial, prepare 9.5 g of the 1-tetralone and 3-phenoxytoluene with a weight ratio of 1:1. Separate out the quantum dots from the solution with acetone, centrifuge and obtain a solid of quantum dots. Weigh 0.5 g the solid of the quantum dots from the glove box, add to the solvent system in the vial, stir and mix. Keep stirring at a temperature of 60° C. until the quantum dots fully dispersed, cool to the room temperature. Membrane Filtrate the quantum dot solution obtained through a 0.2 μm PTFE filter. Seal and store.

### Embodiment 8: Preparing the Printing Ink with the Quantum Dots Containing a Mixture of 1-Tetralone and Acetophenone

Put a stirrer into a vial, clean it up before transferring to a glove box. In the vial, prepare 9.5 g 1-tetralone and acetophenone (with a weight ratio of 9:1). Separate out the quantum dots from the solution with acetone, centrifuge and obtain a solid of quantum dots. Weigh 0.5 g the solid of the quantum dots from the glove box, add to the solvent system in the vial, stir and mix. Keep stirring at a temperature of 60° C. until the quantum dots fully dispersed, cool to the room temperature. Membrane Filtrate the quantum dot solution obtained through a 0.2 μm PTFE filter. Seal and store.

### Embodiment 9: Preparing the Printing Ink with the Quantum Dots Containing a Mixture of 3-Phenoxytoluene and 1-Methoxynaphthalene

Put a stirrer into a vial, clean it up before transferring to a glove box. In the vial, prepare 9.5 g of 3-phenoxytoluene and 1-methoxynaphthalene (with a weight ratio of 9:1). Separate out the quantum dots from the solution with acetone, centrifuge and obtain a solid of quantum dots. Weigh for 0.5 g the solid of the quantum dots from the glove box, add to the solvent system in the vial, stir and mix. Keep stirring at a temperature of 60° C. until the quantum dots fully dispersed, cool to the room temperature. Membrane Filtrate the quantum dot solution obtained through a 0.2 μm PTFE filter. Seal and store.

### Embodiment 10: A Test of the Viscosity and the Surface Tension

The viscosity of the printing ink with the quantum dots was measured by a DV-IPrime Brookfield rheometer; the surface tension of the printing ink with the quantum dots was measured by a SITAbubble pressure tensiometer.

From the tests described above, the viscosity of the printing ink with the quantum dots obtained in the example 5 is 9.3±0.3 cPs, the surface tension is 38.1±0.1 dyne/cm.

From the tests described above, the viscosity of the printing ink with the quantum dots obtained in the example 6 is 6.7±0.3 cPs, the surface tension is 33.1±0.1 dyne/cm.

From the tests described above, the viscosity of the printing ink with the quantum dots obtained in the example 7 is 6.5±0.3 cPs, the surface tension is 35.1±0.1 dyne/cm.

From the tests described above, the viscosity of the printing ink with the quantum dots obtained in the example 8 is 4.3±0.3 cPs, the surface tension is 37.3±0.1 dyne/cm.

From the tests described above, the viscosity of the printing ink with the quantum dots obtained in the example 9 is 6.3±0.3 cPs, the surface tension is 34.9±0.1 dyne/cm.

Using the above prepared printing ink containing quantum dots based on the solvent system of arylketone or arylether, through a method of ink-jet printing, the functional layers in the quantum dot light-emitting diode may be prepared, including the light-emitting layer and the charge transport layer, the specific steps are as follows.

Put the ink containing the quantum dots into an ink tank, and mount the ink tank onto an ink jet printer such as a Dimatix Materials Printer DMP-3000 (Fujifilm). Adjust a waveform, a pulse time and a voltage of jetting the ink, before achieving a best of the ink jetting, and a stability of the ink-jetting range. When preparing a QLED device with a quantum dot film as a light-emitting layer, the following technical solution is adopted: The substrate of the QLED is 0.7 mm-thick glass sputtered with an indium tin oxide (ITO) electrode pattern. On the ITO, a pixel definition layer is patterned to form a plurality of holes for depositing the printing ink inside. Followed by obtaining a HIL/HTL film through ink-jet printing the HIL/HTL material into the holes, and removing the solvent by drying under a high temperature in a vacuum environment. Followed by ink-jet printing the printing ink containing the light emitting quantum dots onto the HIL/HTL film, and removing the solvent by high-temperature drying in a vacuum environment to obtain a quantum dots light-emitting layer film. Followed by ink-jet printing the printing ink containing the quantum dots having the electron transport property onto the light-emitting layer film, and removing the solvent by high-temperature drying in a vacuum environment, to form an electron transport layer (ETL). When using the organic electronic transmission materials, the ETL may also be formed by vacuum thermal evaporation. Then, the Al cathode is formed by vacuum thermal evaporation, and finally the QLED device preparation is completed and packaged.

It should be understood that the above embodiments disclosed herein are exemplary only and not limiting the scope of this disclosure. Without departing from the spirit and scope of this invention, other modifications, equivalents, or improvements to the disclosed embodiments are obvious to those skilled in the art and are intended to be encompassed within the scope of the present disclosure.

What is claimed is:

1. A printing ink, comprising an inorganic nano-material, and at least one aryl ketone-based or aryl ether-based organic solvent, wherein, the aryl ketone-based or aryl ether-based organic solvent has a boiling point over 200° C., and a viscosity in a range of 1 cPs to 100 cPs at 25° C., the aryl ketone-based or aryl ether-based organic solvent is able to be evaporated from a solvent system and forming an inorganic nano-material film.

2. The printing ink according to claim 1, wherein the aryl ketone-based or aryl ether-based organic solvent has a surface tension in a range of 19 dyne/cm to 50 dyne/cm at 25° C.

3. The printing ink according to claim 1, wherein the aryl ketone-based or aryl ether-based organic solvent has a structure shown as a general formula (I) and (II), respectively: 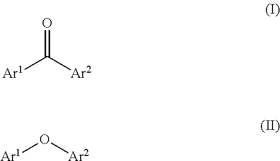
wherein, an Ar¹ and an Ar² may be identical or different, and each is a substituted or unsubstituted aryl or heteroaryl ring system having 5 to 40 ring atoms;
the Ar¹ and the Ar² may be different, and one of them is a substituted or unsubstituted aryl or heteroaryl ring system having 5 to 40 ring atoms, another is a linear alkyl, alkoxy or thioalkoxy group having 1 to 20 carbon atoms, or a branched or cyclic alkyl, alkoxy or thioalkoxy group or a silyl group having 3 to 20 carbon atoms, or a substituted keto group having 1 to 20 carbon atoms, an alkoxycarbonyl group having 2 to 20 carbon atoms, an aryloxycarbonyl group having 7 to 20 carbon atoms, a cyano group (—CN), a carbamoyl group (—C(═O)NH₂), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF₃ group, a Cl, a Br, a F, a crosslinkable group, or a combination thereof.

4. The printing ink according to claim 1, wherein the substituted or unsubstituted aryl or heteroaryl group, that both the Ar¹ and the Ar² in the general formulas (I) and (II) are selected from, has a structure shown as a general formula below: 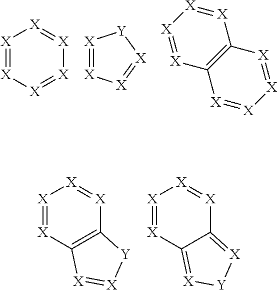
wherein,
X is CR¹ or N;
Y is selected from CR²R³, SiR²R³, NR² or, C(═0), S, or O;
R¹, R², R³ is a H, a D, or a linear alkyl, alkoxy or thioalkoxy group having 1 to 20 carbon atoms, or a branched or cyclic alkyl, alkoxy or thioalkoxy group or a silyl group having 3 to 20 carbon atoms, or a substituted keto group having 1 to 20 carbon atoms, an alkoxycarbonyl group having 2 to 20 carbon atoms, an aryloxycarbonyl group having 7 to 20 carbon atoms, a cyano group (—CN), a carbamoyl group (—C(═O)NH₂), a haloformyl group (—C(═O)—X, wherein X represents a halogen atom), a formyl group (—C(═O)—H), an isocyano group, an isocyanate group, a thiocyanate group or an isothiocyanate group, a hydroxyl group, a nitro group, a CF₃ group, a Cl, a Br, a F, a crosslinkable group or a substituted or unsubstituted aryl or heteroaryl ring system having 5 to 40 ring atoms, or an aryloxy or a heteroaryloxy group having 5 to 40 ring atoms, or a combination thereof, wherein one or more groups of R¹, R², R³ may form a mono or polycyclic aliphatic or aryl ring system by themselves or rings bonded with the groups.

5. The printing ink according to claim 1, wherein the organic solvent is a single aryl ketone solvent or a mixture of a plurality of aryl ketone solvents or a mixture of aryl ketone solvents and other solvents; or the organic solvent is a single aryl ether solvent, or a mixture of a plurality of aryl ether solvents, or a mixture of aryl ether solvents and other solvents; or the organic solvent is a mixture of aryl ketone solvents and aryl ether solvents or a mixture further mixed with other solvents.

6. The printing ink according to claim 1, wherein the organic solvent of the aryl ketone is 1-tetralone, 2-tetralone, acetophenone, propiophenone, benzophenone and a plurality of derivatives thereof.

7. The printing ink according to claim 1, wherein the organic solvent of the aryl ether is 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl) benzene, 1,3-dipropylbenzene, 2,5-dimethoxytoluene, dibenzyl ether, 1,2-dimethoxybenzene, glycidic phenyl ether.

8. The printing ink according to claim 1, wherein the aryl ketone-based organic solvent is a tetralone or a solvent comprising at least 50% by weight of the tetralone and at least one other additional solvent.

9. The printing ink according to claim 1, wherein the aryl ether-based organic solvent is a 3-phenoxytoluene or a solvent comprising at least 50% by weight of the 3-phenoxytoluene and at least one other additional solvent.

10. The printing ink according to claim 1, wherein the inorganic nano-material is a quantum dot material, that is, the particle diameter thereof has a monodisperse size distribution, and a shape thereof may be selected from a plurality of different forms, including a sphere, a cube, a rod or a branched structure.

11. The printing ink according to claim 10, wherein at least one luminescent quantum dot material is comprised, with a luminescence wavelength between 380 nm and 2500 nm.

12. The printing ink according to claim 1, wherein the at least one inorganic nano-material is a binary or multiple semiconductor compound or a mixture thereof in Group IV, Group II-VI, Group II-V, Group III-V, Group III-VI, Group IV-VI, Group I-III-VI, Group II-IV-VI, Group II-IV-V of the Periodic Table.

13. The printing ink according to claim 1, wherein the at least one inorganic nano-material is a perovskite nanoparticle material, specifically, a perovskite nanoparticle material, a metal nanoparticle material or a metal oxide nanoparticle material having a luminescent property, or a mixture thereof.

14. The printing ink according to claim 1, wherein at least one organic functional material is further comprised, the organic functional material may be selected from a hole injection material (HIM), a hole transport material (HTM), an electron transport materials (ETM), an electron injection material (EIM), an electrons blocking material (EBM), a hole blocking material (HBM), a light emitter (Emitter) and a host material (Host).

15. The printing ink according to claim 1, wherein a weight ratio of the inorganic nano-material is 0.3%˜70%, a weight ratio of the organic solvent comprised based on aryl ketone or aryl ether is 30%˜99.7%.

16. An electronic device comprises a functional layer printed by the printing ink according to claim 1, wherein the aryl ketone-based or aryl ether-based organic solvent may be evaporated from the solvent system, and forming a film comprising the inorganic nano-material.

17. The electronic device according to claim 16, wherein the electronic device is selected from a quantum dot light emitting diode (QLED), a quantum dot photovoltaic cell (QPV), a quantum dot light emitting electrochemical cell (QLEEC), a quantum dot field-effect transistor (QFET), a quantum dot light emitting field-effect transistor, a quantum dot Laser, a quantum dot sensor.
